(19) United States
(12) Patent Application Publication
(10) Pub. No.: US 2017/0319395 A1
(43) Pub. Date: Nov. 9, 2017

(54) VENOUS COMPRESSION ORTHOSIS WITH VARIABLE PRESSURE PROFILE

(71) Applicant: LABORATOIRES INNOTHERA, Arcueil (FR)

(72) Inventors: François CROS, Ivry Sur Seine (FR); Amina OUCHENE, Creteil (FR); Joël MATHIEU, Eloyes (FR)

(73) Assignee: LABORATOIRES INNOTHERA, Arcueil (FR)

(21) Appl. No.: 15/589,012

(22) Filed: May 8, 2017

(30) Foreign Application Priority Data
May 6, 2016 (FR) 16 54103

(51) Int. Cl. A61F 13/08; D04B 21/20

(52) U.S. Cl. CPC A61F 13/08; D04B 21/207

(57) ABSTRACT

An elastic venous compression orthosis indicated in cases of vein disorders of a lower limb of a patient, the lower limb having an ankle, a calf and a thigh, the orthosis having an ankle part for enclosing the ankle and a calf part for enclosing the calf, the orthosis having a textile pressure profile such that the textile pressure measured at the ankle is less than 10 mmHg, and the textile pressure measured at the calf is less than 35 mmHg and greater than 25 mmHg.

### TECHNICAL FIELD

The invention relates to an elastic venous compression orthosis, hereinafter “orthosis”, indicated in cases of vein disorders of a lower limb of a patient.

### PRIOR ART

Elastic venous compression orthoses, formerly known as “retention stockings (or hoses)” or “retention tights”, are textile medical devices producing a therapeutic effect through compression of the lower limbs, unlike “support stockings” (or even “support hoses” or “anti-fatigue stockings”) and “fashion stockings”, which are not medical devices with a therapeutic purpose.

To permit strong compression of the lower limbs, the elastic venous compression orthoses are traditionally made from a knitted mesh with incorporation of an elastic weft thread, generally a covered elastane.

The orthosis is put onto the lower limb of the patient to be treated, as far as a position of use. The restoring force of the elastic fibres then exerts a compression.

The mesh and the threads, and the dimensioning of the rows of meshes, are chosen in such a way as to apply predetermined textile pressures at different altitudes of the lower limb, for example at the height of the ankle, at the start of the calf, at the level of the calf, at the popliteal fossa, etc., as far as the top of the thigh, said altitudes being conventionally designated B to G.

These different textile pressures are defined for each class by reference to metrological jigs such as the leg model of French standard NF G 30-102 part B, annex B, corresponding to the “Hohenstein” leg model according to the German standard RAL-GZ 387, or as defined in the experimental European standard XP ENV 12718:2001.

The measurement points of the lower limb are defined by the European Commission for the Standardization of Medical Compression Stockings (C. Gardon-Mollard, A. Ramelet, La compression médicale, Ed. Masson, 2^{nd} edition).

As is shown in FIG. 2, the altitudes of the lower limb are defined by the morphological reference system. They have been shown using the standard notation:
B: ankle, at the point of its smallest circumference;
B1: junction between the Achilles tendon and the calf muscles;
C: calf, at the point of its greatest circumference;
D: just below the tibial tuberosity (that is to say just below the knee);
E: at the centre of the knee cap and above the back of the knee (that is to say at the level of the popliteal fossa);
F: at the middle of the thigh; and
G: at the top of the thigh.

The calf is the limb segment situated between levels B1 and D, and the ankle is the limb segment extending from level B to the instep.

The textile pressure profile, that is to say the change, along an orthosis, of the textile pressure exerted by the orthosis fitted on the morphological jig, determines its therapeutic efficacy. WO2010/037609 describes examples of textile pressure profiles.

To promote venous return in an orthostatic situation, the textile pressure profile is traditionally a degressive one.

To promote venous return when the patient is walking, the textile pressure profile is, by contrast, progressive in an upward direction from the ankle. Indeed, such a profile improves the efficacy of the “calf musculo-aponeurotic pump” (CMAP) which, by virtue of the physiological muscle contractions of the calf, causes a centripetal venous flush in the muscular and deep venous network.

Moreover, in order to improve compliance, an orthosis must be practical and comfortable.

There is a constant need to improve the efficacy and comfort of the orthoses.

An object of the invention is to meet this need at least in part.

### SUMMARY OF THE INVENTION

According to the invention, this object is achieved by means of an elastic venous compression orthosis indicated in cases of vein disorders of a lower limb of a patient, said orthosis having a textile pressure profile such that:
the textile pressure measured at the ankle, that is to say at altitude B, is less than 10 mmHg (i.e. substantially 13.3 hPa, although mmHg is customarily used as the unit of textile pressure measurement in the field of phlebology and medical compression), preferably less than 7 mmHg (i.e. substantially 9.3 hPa), preferably less than 5 mmHg (i.e. substantially 6.7 hPa), and preferably greater than 3 mmHg (i.e. 4 hPa), and
the textile pressure measured at the calf, that is to say at altitude C, is less than 35 mmHg (i.e. substantially 46.7 hPa), preferably less than 33 mmHg (i.e. substantially 44 hPa), preferably less than 30 mmHg (i.e. 40 hPa), and greater than 25 mmHg (i.e. substantially 33.3 hPa), preferably greater than 28 mmHg (i.e. substantially 37.3 hPa).

The inventors have found that an orthosis according to the invention is effective and particularly comfortable. In particular, it is easy to put on, and the therapeutic efficacy is remarkable.

An orthosis according to the invention may also have one or more of the following optional and preferred features:
the textile pressure measured at the middle of the thigh, that is to say at altitude F, is less than 10 mmHg (i.e. substantially 13.3 hPa), preferably less than 7 mmHg (i.e. substantially 9.3 hPa), preferably less than 5 mmHg (i.e. substantially 6.7 hPa), and preferably greater than 3 mmHg (i.e. 4 hPa);
the ankle part and/or the calf part have, at any given altitude, a constant structure along the circumference of said orthosis at said altitude;
the mesh height of the calf part is greater than 400, greater than 450 or greater than 500, and the mesh height of the ankle part is greater than 700, greater than 750 or greater than 800;
the mesh height of the ankle part is greater than the mesh height of the calf part, the increase preferably being progressive.

### BRIEF DESCRIPTION OF THE FIGURES

Other features and advantages of the invention will become clearer from reading the following detailed description and from examining the attached drawing, in which:

FIG. 1 shows an orthosis according to the invention fitted on a lower limb of a patient;

FIG. 2 shows a schematic view of the standard morphological reference system; and

FIGS. 3 and 4 illustrate the test results.

### DEFINITIONS

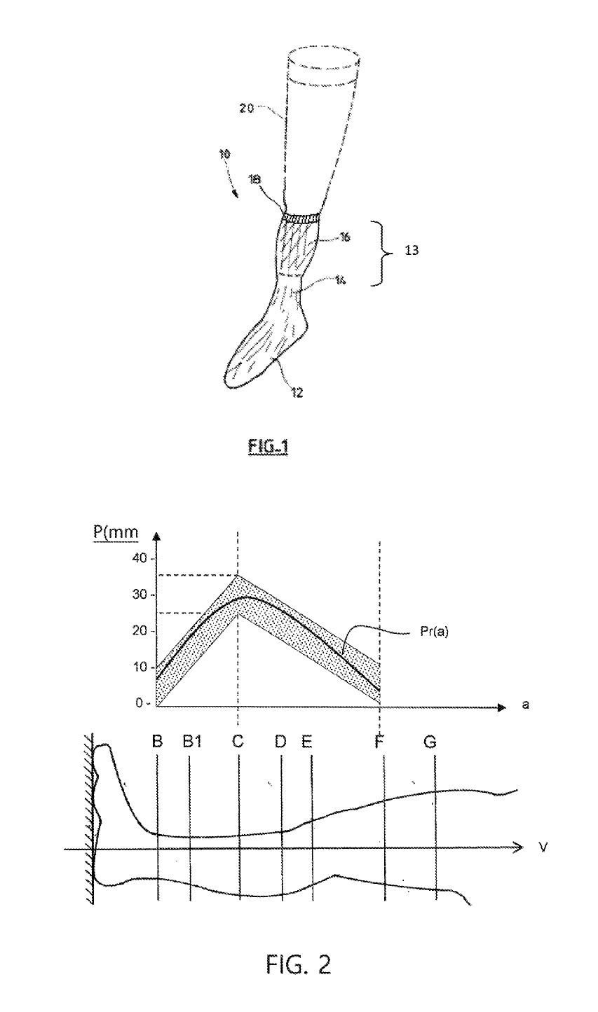

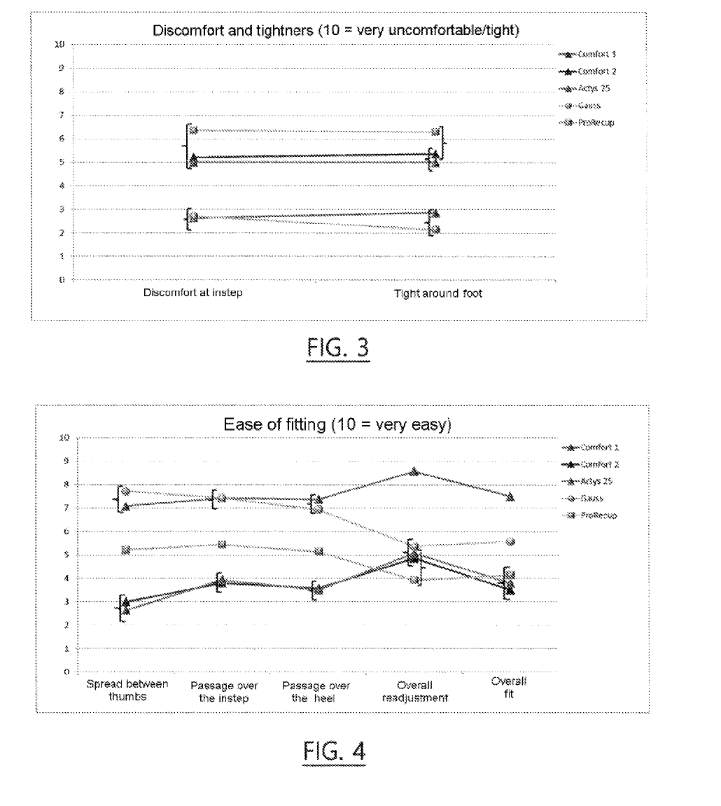

The terms “compression” and “retention” define very different effects, although they are sometimes mixed up in everyday language.
“Compression” is the effect produced by an elastic orthosis, both at rest and during effort, on a limb segment, as a result of the more or less strong restoring forces of the elastic fibres of this orthosis. These forces act on the limb in an almost constant manner: at rest, the compression is present at the nominal pressure value, and, during effort, the effect of this compression is slightly increased by the contraction of the muscle masses.
Conversely, “retention” is the effect produced by an orthosis that acts in a differentiated manner (effort/rest) on a limb segment, under the action of a structure considered as being inelastic (but deformable), for example a non-elastic bandage, also referred to as “short-stretch bandage”. At rest, this type of bandage exerts a low pressure, or even no pressure. However, during muscle contraction, it works against the local volume increases of the muscle, which comes into abutment with the non-elastic structure, the pressure thus being strongly increased. Retention is thus effective and active during effort, and almost inactive at rest. In the scientific literature on this subject, it is customary to consider that an orthosis is retentive, or “rigid”, when it produces an increase of at least 10 mmHg (13 hPa) per centimetre of increase of the limb circumference at the point located at the junction between the Achilles tendon and the calf muscles. Here, “rigidity” is understood within the meaning of the definition of the European Pre-Standard XP ENV 12718:2001, i.e. as meaning “the increase of compression per centimetre of increase of leg circumference, expressed in hectopascals per centimetre and/or in millimetres of mercury per centimetre”.
“Altitude” or “level” corresponds to a level in the vertical direction V when the orthosis is worn by a patient standing up straight, as shown in FIG. 2.
The French standard NF G 30-102, part B, defines and provides the protocol for measuring a pressure called the “retention pressure”. This standard applies likewise to the measurement of compression pressures.
To this end, this standard uses a leg model of the “Hohenstein” type composed of cone segments. At a defined altitude, the circumference of an orthosis fitted on such a leg model, that is to say along a contour corresponding to a horizontal section of the limb, is therefore circular.
Traditionally, at a defined altitude, the structure of the orthosis is constant along its circumference. Since the cross section is circular, the retention pressure measured at a point on this circumference is thus constant, irrespective of the point considered.
Generically, “textile pressure” designates the pressure within the meaning of the standard NF G30102, that is to say the pressure that a taut textile exerts on a template of circular cross section.

### DETAILED DESCRIPTION

In FIG. 1, reference sign 10 generally designates an orthosis according to the invention.

The orthosis 10 is shaped to cover the ankle and the calf, that is to say it extends at least from altitude B to altitude D.

The orthosis 10, which is knitted, has the general shape of a deformable sheath that is to be fitted on a lower limb of a patient. Of anatomical shape, the orthosis comprises in succession from the end of the foot:
optionally, a foot part 12 enclosing the foot;
a leg part 13 having an ankle part 14 enclosing the ankle and a calf part 16 enclosing the calf;
optionally, a thigh part 20 enclosing the thigh; and
optionally, a knitted end part called the “ribbing” 18, which ends below the knee.

The orthosis 10 shown extends to a level situated below the knee, in the case where the orthosis is a “half-hose” (or “knee sock”).

The configuration in the form of a sock is not limiting, and the orthosis 10 may also be produced in the form of a “thigh stocking”, the leg part 13 being continued by the compressive thigh part 20. The orthosis 10 may also be produced in the form of tights and/or without a foot part 12 (stocking or tights of the “open foot” type).

The various adjoining parts of the orthosis 10 are preferably knitted continuously on a circular machine, according to conventional methods. Advantageously, the production of the orthosis 10 does not require any preparatory step for assembling the various parts, except for the operations of sewing on the foot part 12, if the latter is present.

The structure of the orthosis traditionally comprises a network of meshes that are produced by means of a knit thread, with a weft thread being interlaced between said meshes.

The orthosis 10 is traditionally produced by knitting a knit thread and a weft thread, the size and nature of the knit and weft threads and the mesh structure being chosen so as to exert in the circumferential direction, after the orthosis has been placed on the lower limb, an elastic restoring force that is able to produce a compression of the limb at the desired therapeutic pressure level.

In a preferred embodiment, all the parts of the orthosis are compressive.

Depending on the altitude, the knitted mesh can have a more or less tight texture.

Preferably, the weft thread is elastic, preferably a double-coated thread of elastane.

Adapting the elastic restoring force in order to obtain a defined textile pressure profile does not pose any particular difficulty to a person skilled in the art.

The graph shown at the top of FIG. 2 depicts the pressure zone (shaded zone) in which the textile pressure profile of an orthosis according to the invention is preferably contained.

The curve Pr(a) represents an example of a textile pressure profile giving the textile pressure P as a function of the altitude a.

The textile pressure profile may have points of inflexion or, as is shown, may vary in a continuous manner.

It may have pressure stages, the textile pressure being constant over part of the height of the orthosis, for example over more than 1 cm, more than 3 cm or more than 5 cm. By contrast, the textile pressure profile may vary over the entire height of the orthosis.

By way of example, the calf part can be produced by knitting with four feeds:
1st feed=plain jersey stitch all needles with 2 ends of (lycra 17 dtex type175C air-coated with semi-matt polyamide 66 1/44/13 FTS);
2nd feed=charge 1 needle in 2 with 1 end of (lycra 940 dtex type 136C double-coated with semi-matt polyamide 66 1/78/23 FT);
3rd feed=plain jersey stitch all needles with 2 ends of (lycra 17 dtex type 175C air-coated with semi-matt polyamide 66 1/44/13 FTS);
4th feed=charge 1 needle in 2 with 1 end of (lycra 570 dtex type 902C double-coated with semi-matt polyamide 66 1/22/7 FT).

The mesh height for the calf part may be 500.

The same knitting can be applied for the ankle part, by increasing the mesh height to 800. The increase in the mesh height is preferably progressive, for example in the region located between altitudes B and B1.

Tests have shown that textile pressure profiles according to the invention lead to remarkable performance in terms of efficacy and comfort.

In particular, a panel of 7 women (whose ankle circumferences were between 19 and 22 cm), who were instructed and trained in the techniques of sensory analysis, evaluated 9 descriptors defining the ease of fitting, the discomfort felt on movement, and the feeling of tightness of 5 stockings with degressive or progressive compression using a methodology developed by IFTH in collaboration with Laboratoires Innothera on the basis of the sensory analysis standards ISO 11035, ISO 11036, BP X10 040 and BP X10 041.

The aim was to compare the ease of fitting, the discomfort felt on movement and the feeling of tightness of degressive compression stockings (classes 1 to 3) and products delivering a progressive compression.

The results are set out in the attached figures.

In these figures, the bars or braces denote statistically equivalent products according to the Duncan test at 5%. In the IFTH report, the code/product connections are:
A: Innothera Comfort 1
B: Innothera Comfort 2
C: Innothera Gauss 5-30 (progressive), according to the invention
D: BV Sport ProRecup (progressive)
E: Innothera Actys 25

The tests show that the product that is easiest to fit overall is Comfort 1, then Gauss. These are followed with statistical equivalence by ProRecup, Acys 25 and Comfort 2. The products causing the least discomfort during movement and the least amount of tightness are Comfort 1 and Gauss, with statistical equivalence. By contrast, with statistical equivalence, Comfort 2, Actys 25 and then ProRecup, in terms of comfort for the wearer.

The therapeutic efficacy of the Gauss product is greater than that of the Comfort 1 product. The latter is in fact a class 1 product, indicated for the early stages of vein disorders. The Gauss product advantageously has the efficacy of a class 3 product.

An orthosis according to the invention is suitable for the therapeutic treatment of chronic disorders of the veins, in particular for the treatment of venous insufficiency below or equal to C3 in the CEAP classification (C: clinical manifestation; E: etiologic factors; A: anatomic distribution; P: pathophysiologic dysfunction).

Of course, the invention is not limited to the embodiments described and shown, which are provided for illustrative purposes only.

1. Elastic venous compression orthosis, which is knitted and has the shape of a deformable sheath and which is indicated in cases of vein disorders of a lower limb of a patient, said lower limb having an ankle, a calf and a thigh, said orthosis having an ankle part for enclosing the ankle and a calf part for enclosing the calf, said orthosis having a textile pressure profile such that:
the textile pressure measured at the ankle is less than 10 mmHg, and
the textile pressure measured at the calf is less than 35 mmHg and greater than 25 mmHg,
the textile pressure being the pressure within the meaning of the standard NF G30102.

2. Orthosis according to claim 1, in which:
the textile pressure measured at the ankle is less than 7 mmHg, and
the textile pressure measured at the calf is less than 33 mmHg and greater than 28 mmHg.

3. Orthosis according to claim 1, in which:
the textile pressure measured at the ankle is less than 5 mmHg, and
the textile pressure measured at the calf is less than 30 mmHg.

4. Orthosis according to claim 1, in which:
the textile pressure measured at the ankle is greater than 3 mmHg.

5. Orthosis according to claim 1, said orthosis having a thigh part for enclosing the thigh, said orthosis having a textile pressure profile such that:
the textile pressure measured at the middle of the thigh is less than 10 mmHg.

6. Orthosis according to claim 5, in which:
the textile pressure measured at the middle of the thigh is less than 5 mmHg.

7. Orthosis according to claim 6, in which:
the textile pressure measured at the middle of the thigh is less than 3 mmHg.

8. Orthosis according to claim 1, in which the mesh height of the ankle part is greater than the mesh height of the calf part.

9. Orthosis according to claim 1, chosen from the group composed of a half-hose, a thigh stocking and tights.
